(19) United States
(12) Patent Application Publication
(10) Pub. No.: US 2026/0080708 A1
(43) Pub. Date: Mar. 19, 2026

(54) MULTI-STAGE FRAMEWORK FOR EXTRACTING KEY/VALUE PAIRS FROM IMAGES

(71) Applicant: The Manufacturers Life Insurance Company, Toronto (CA)

(72) Inventors: Gareth Sharpe, Stratford (CA); Gopi Balasingam, Markham (CA)

(21) Appl. No.: 19/310,740

(22) Filed: Aug. 26, 2025

Related U.S. Application Data
(60) Provisional application No. 63/696,816, filed on Sep. 19, 2024.

(51) Int. Cl. G06V 30/416; G06F 40/103; G06V 10/20; G06V 10/40; G06V 10/764; G06V 10/776; G06V 10/98; G06V 30/12; G06V 30/16; G06V 30/18; G06V 30/19; G06V 30/413; G06V 30/42; G16H 10/00

(52) U.S. Cl. CPC G06V 30/416; G06F 40/103; G06V 10/40; G06V 10/764; G06V 10/776; G06V 10/993; G06V 30/133; G06V 30/18; G06V 30/1916; G06V 30/19173; G06V 30/413; G06V 30/42; G16H 10/00; G06V 10/20; G06V 30/16; G06V 2201/03

(57) ABSTRACT

Systems and methods for processing document images using large language models to extract a key/value pair. The method includes a four-stage framework: (1) Image Quality Evaluation, assessing image attributes like text legibility and sharpness; (2) Image Classification, categorizing documents into predefined types; (3) Key/Value Pair Extraction, identifying relevant data fields; and (4) Extraction Evaluation, assigning confidence scores based on one or more predetermined criteria. The process employs prompt engineering to configure structured prompts for guiding the model at each stage. Outputs, including confidence scores and extracted data, are formatted for integration with downstream workflows, enabling applications in claims processing, invoicing, and other document-centric tasks.

### TECHNICAL FIELD

The present disclosure relates to systems and methods for leveraging machine learning models to extract key/value pairs from images.

### BACKGROUND

Using machine learning models to extract meaningful content from images, particularly in the form of text and structured data, is a technical challenge relevant to multiple industries. This process involves digitizing physical documents and automating workflows that extract structured data from a variety of document types, such as invoices, forms, and reports. The differences in text layout, alignment, and visual presentation between documents can make it difficult to achieve consistent and accurate results, especially when processing large volumes of documents presented in various formats. Factors such as varying font sizes, alignment discrepancies, and variations in image quality further complicate the extraction process. These complexities are relevant in scenarios where the extracted data informs subsequent workflows or decision-making processes. Thus, ensuring the accuracy and integrity of such data would be beneficial for effective automation.

### SUMMARY

According to a first aspect, there is provided a method for extracting a key/value pair from an image file, the method comprising: receiving the image file, wherein the image file comprises an input image representative of a document, and wherein the input image depicts one or more key/value pairs, wherein each of the key/value pairs comprises a key and an associated value as content within the document; inputting the input image to one or more large language models (LLMs); generating an image quality score indicating suitability of the input image for Optical Character Recognition (OCR) by inputting an image quality prompt comprising one or more image quality criteria to the one or more LLMs; determining that the image quality score satisfies an image quality threshold; applying OCR to the input image to obtain OCR output data comprising textual and structural features; classifying the input image into a document type selected from a plurality of predetermined types by inputting a classification prompt including the textual and structural features to the one or more LLMs; extracting the one or more key/value pairs from the OCR output data by inputting an extraction prompt including an extraction rule determined by the classified document type to the one or more LLMs; and generating a confidence score for the one or more key/value pairs by inputting an evaluation prompt to the one or more LLMs, the evaluation prompt including one or more textual evaluation criteria including visual consistency between the one or more key/value pairs and part of the input image containing the one or more key/value pairs.

In some embodiments, the method may further comprise: arranging the one or more key/value pairs and the confidence score in a structured document; and storing the structured document.

In some embodiments, the one or more image quality criteria may comprise at least one of text legibility, image sharpness, contrast, noise level, or text alignment.

In some embodiments, the image quality score may be an average of the image quality scores corresponding to the one or more image quality criteria.

In some embodiments, the document may be a medical-related document, and the plurality of predetermined types includes one or more of an invoice, a prescription, or a claim form.

In some embodiments, the textual and structural features of the OCR output data may comprise at least one of a keyword or a layout element identified within the OCR output data.

In some embodiments, the layout element may comprise one or more of a checkbox, a table, a header, a section break, or an angle of rotation of the input image.

In some embodiments, the confidence score may be an average of confidence scores associated with all of the key/value pairs.

In some embodiments, the method may further comprise, removing the key/value pair having a blank value field.

In some embodiments, the method may further comprise preprocessing the input image before applying the OCR by performing at least one of cropping, rotating, resizing, or enhancing a contrast of the input image.

In some embodiments, the preprocessing may be performed before generating the image quality score.

In some embodiments, the one or more textual evaluation criteria may further comprise relevance between the one or more key/value pairs and the classified document type, wherein the method further comprises determining the relevance by assessing whether the extracted key/value pairs correspond to fields expected for the classified document type based on a predefined rule.

In some embodiments, the one or more textual evaluation criteria may further comprise visual clarity, the visual clarity being derived from the image quality score for the part of the input image containing the one or more key/value pairs.

In some embodiments, the one or more textual evaluation criteria may comprise weights applied to the visual consistency, the relevance, and the image clarity such that the confidence score is a weighted evaluation of the textual evaluation criteria.

In some embodiments, the weights applied to the visual consistency, the relevance, and the image clarity may be 50%, 25%, and 25%, respectively.

In some embodiments, the image quality prompt may comprise: the one or more image quality criteria and corresponding definitions; and a natural language request to estimate an image quality of the input image on a numerical scale as the image quality score.

In some embodiments, the classification prompt may comprise: the textual and structural features; the plurality of predetermined types; the one or more classification criteria and corresponding definitions; and a natural language request to classify the input image into one of the plurality of predetermined types based on the textual and structural features.

In some embodiments, the extraction prompt may further comprise: guidance for handling different types of the textual and structural features; and a natural language request to format the extracted the one or more key/value pairs into a structured output.

In some embodiments, the evaluation prompt may comprise: the one or more key/value pairs; the one or more textual evaluation criteria and corresponding definitions; and a natural language request to evaluate the one or more key/value pairs on a numerical scale as the confidence score.

In some embodiments, the generation of the image quality score, the classification of the input image, and the extraction of the one or more key/value pairs may be performed by a first LLM, and the generation of the confidence score may be performed by a second LLM, distinct from the first LLM, the second LLM acting as a judge.

According to a second aspect, there is provided a non-transitory computer-readable medium storing computer-executable instructions that, when executed by one or more processing units, cause the one or more processing units to perform a method for extracting a key/value pair from an image file, the method comprising: receiving the image file, wherein the image file comprises an input image representative of a document, and wherein the input image depicts one or more key/value pairs, wherein each of the key/value pairs comprises a key and an associated value as content within the document; inputting the input image to one or more large language models (LLMs); generating an image quality score indicating suitability of the input image for Optical Character Recognition (OCR) by inputting an image quality prompt comprising one or more image quality criteria to the one or more LLMs; determining that the image quality score satisfies an image quality threshold; applying OCR to the input image to obtain OCR output data comprising textual and structural features; classifying the input image into a document type selected from a plurality of predetermined types by inputting a classification prompt including the textual and structural features to the one or more LLMs; extracting the one or more key/value pairs from the OCR output data by inputting an extraction prompt including an extraction rule determined by the classified document type to the one or more LLMs; and generating a confidence score for the one or more key/value pairs by inputting an evaluation prompt to the one or more LLMs, the evaluation prompt including one or more textual evaluation criteria including visual consistency between the one or more key/value pairs and part of the input image containing the one or more key/value pairs.

According to a third aspect, there is provided a system for processing an input image of a document, the system comprising: a processing unit; a memory communicatively coupled to the processing unit, the memory storing computer-executable instructions that, when executed by the processing unit, cause the processing unit to perform a method for extracting a key/value pair from an image file, the method comprising: receiving the image file, wherein the image file comprises an input image representative of a document, and wherein the input image depicts one or more key/value pairs, wherein each of the key/value pairs comprises a key and an associated value as content within the document; inputting the input image to one or more large language models (LLMs); generating an image quality score indicating suitability of the input image for Optical Character Recognition (OCR) by inputting an image quality prompt comprising one or more image quality criteria to the one or more LLMs; determining that the image quality score satisfies an image quality threshold; applying OCR to the input image to obtain OCR output data comprising textual and structural features; classifying the input image into a document type selected from a plurality of predetermined types by inputting a classification prompt including the textual and structural features to the one or more LLMs; extracting the one or more key/value pairs from the OCR output data by inputting an extraction prompt including an extraction rule determined by the classified document type to the one or more LLMs; and generating a confidence score for the one or more key/value pairs by inputting an evaluation prompt to the one or more LLMs, the evaluation prompt including one or more textual evaluation criteria including visual consistency between the one or more key/value pairs and part of the input image containing the one or more key/value pairs.

This summary does not necessarily describe the entire scope of all aspects. Other aspects, features and advantages will be apparent to those of ordinary skill in the art upon review of the following description of specific embodiments.

### BRIEF DESCRIPTION OF THE FIGURES

In the accompanying drawings, which illustrate one or more example embodiments:

FIG. 1A is a schematic diagram illustrating a system for extracting key/value pairs from images, according to an example embodiment.

FIG. 1B is a medical claim form image illustrating multiple key/value pairs to be extracted, according to an example embodiment.

FIG. 2 is a flowchart illustrating a method for extracting key/value pairs from images, according to an example embodiment.

FIG. 3 is a flowchart of specific operations comprising part of the method of FIG. 2.

FIG. 4 is an image quality prompt used to guide image quality evaluation, according to an example embodiment.

FIG. 5 is an output of an image quality evaluation, according to an example embodiment.

FIG. 6 is a classification prompt guiding the categorization of document images into predefined types, according to an example embodiment.

FIG. 7A is an extraction prompt showing instructions for handling OCR results, according to an example embodiment.

FIG. 7B is an extended version of the extraction prompt shown in FIG. 7A.

FIG. 8 is a Pydantic model schema used for heuristic evaluation of extracted key/value pairs, according to an example embodiment.

FIG. 9 is a LLM-as-Judge evaluation process, according to an example embodiment.

FIG. 10 is a bar chart illustrating classification and extraction results for a dataset of document images, according to an example embodiment.

FIG. 11 is a confidence interval plot showing the range of successful classification and extraction rates, according to an example embodiment.

FIG. 12 is a block diagram of an example computer system that may be used to implement the method and system for extracting key/value pairs from images, according to an example embodiment.

### DETAILED DESCRIPTION

The embodiments discussed herein involve or relate to artificial intelligence (AI). AI may involve perceiving, synthesizing, inferring, predicting, and/or generating information using computerized tools and techniques (e.g., machine learning). For example, AI systems may use a combination of hardware and software to rapidly perform complex operations, including perceiving patterns in data, synthesizing outputs, inferring relationships, predicting outcomes, and generating structured information. AI systems often utilize one or more machine learning models, which may be selected or trained to have specific configurations (e.g., model parameters that result from training and various model architectures, as described below). A model's parameters may evolve over time as it learns from input data (e.g., training datasets), enabling the model to refine its capabilities. For example, a dataset can be input into a model to produce an output based on the dataset and the model's architecture and parameters. With additional input (e.g., validation data, reference data, feedback), the model may be further trained (e.g., fine-tuned) to improve its performance over time.

The combination of sophisticated model configurations, extensive datasets, and advanced processing hardware underpins the capabilities of AI systems, allowing them to interpret and process vast amounts of information. These systems can address complex tasks, such as recognizing patterns in image-based data, extracting structured information, and automating workflows that would otherwise require significant manual effort. For example, AI systems can efficiently process documents by analyzing text and structural data, extracting key/value pairs, and categorizing content according to predefined criteria.

As used herein, the term “key/value pairs” refers to structured data representations in which a specific field (the “key”) is associated with a corresponding data entry (the “value”). In the context of document processing, a key may correspond to a label or identifier within the document—such as “Policy Number,” “Patient Name,” or “Date of Service”—while the value is the specific information associated with that key. Key/value pairs enable the extraction and organization of information in a format suitable for structured storage, analysis, or further processing, and may be particularly useful when converting unstructured or semi-structured image-based content into machine-readable outputs.

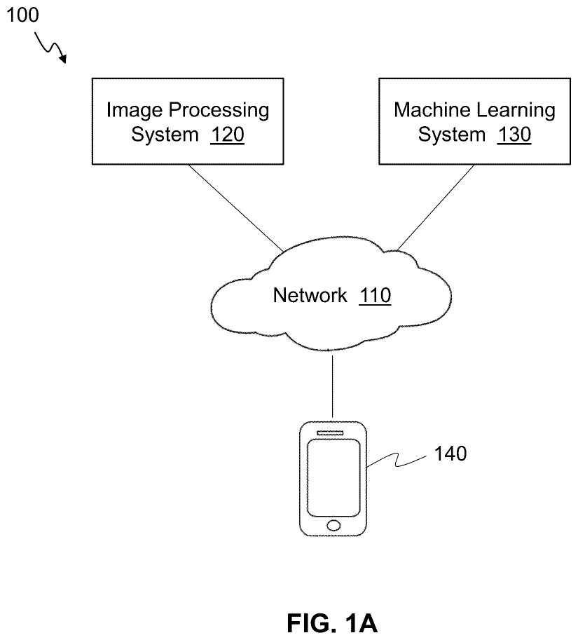

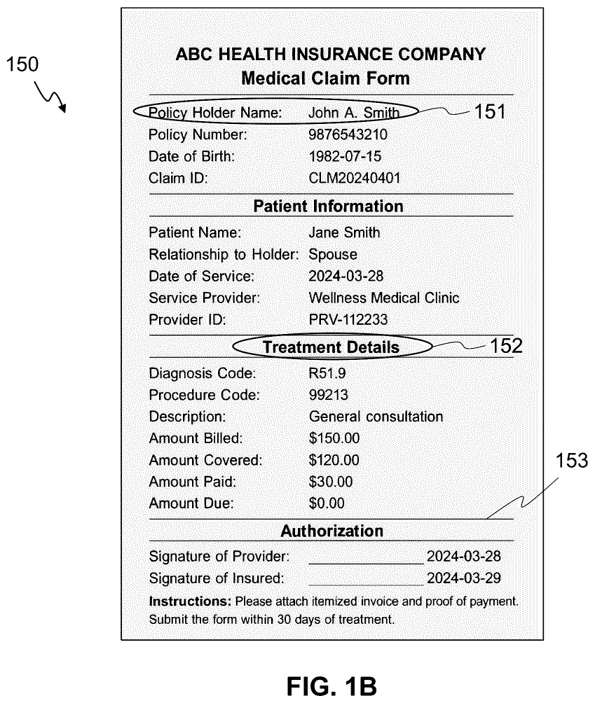

FIG. 1A is a schematic diagram illustrating an example system 100 for extracting key/value pairs from images.

The system 100 may include any number, or any combination of the components shown in FIG. 1A or may include other components or devices that perform or assist in the performance of the system or method consistent with disclosed embodiments. The arrangement of the components of the system 100 shown in FIG. 1A may vary.

FIG. 1A shows the system 100, including an image processing system 120, a machine learning system 130, and a user device 140 communicatively connected together via a network 110. In some embodiments, a user may access and interact with a machine learning model hosted by the machine learning system 130 via a GUI run on the user device 140 to submit requests in relation to one or more images or to manipulate such one or more images. To respond to such requests, the machine learning model may employ the image processing system 120 when processing images according to the requests submitted by the user.

The network 110 may be a wide area network (WAN), local area network (LAN), wireless local area network (WLAN), Internet connection, client-server network, peer-to-peer network, or any other network or combination of networks that would enable the system components to be communicatively linked. The network 110 enables the exchange of information between components of the system 100 such as the image processing system 120, the machine learning system 130, and the user device 140.

The image processing system 120 can be configured to perform the execution of tasks such as image acquisition, analysis, and manipulation. Manipulation may include image enhancement, segmentation, restoration, compression, etc.

The machine learning system 130 may host one or more machine learning models, such as a multimodal large language model (LLM). In some embodiments, the LLM may be based on a transformer architecture and trained on both textual and visual modalities, enabling it to process and reason over combined inputs such as text prompts and image data. The model may incorporate vision encoders, text encoders, and cross-modal attention mechanisms to align and interpret information across different formats. In various implementations, the LLM may be provided by third-party services or platforms, such as OpenAI™ or LLaMA™, or other commercially or publicly available models, and may be accessed via application programming interfaces (APIs) or integrated directly into the machine learning system 130. The choice of model may vary depending on implementation requirements, computational constraints, or licensing considerations. In some embodiments, the machine learning system 130 also manages the training or fine-tuning of at least one machine learning model.

The user device 140 can be any of a variety of device type, such as a personal computer, a mobile device like a smartphone or tablet, a client terminal, etc. In some embodiments, the user device 140 includes at least one monitor or any other such display device. In some embodiments, the user device 140 includes at least one of a physical keyboard, on-screen keyboard, or any other input device through which the user can input text. In some embodiments, the user device 140 allows a user to interact with a GUI (for example a GUI of an application run on or supported by the user device 140) using a machine learning model. For example, while interacting with the GUI, the user of user device 140 may interact with a multimodal LLM that automates, for the user, certain functions of the GUI in cooperation with the image processing system 120 and/or the machine learning system 130.

In some embodiments, the system 100 may further include or interface with one or more image acquisition devices, such as a digital camera on the user device 140 or a separate document scanner (not shown), to capture images of physical documents for processing. The captured image may be transmitted directly to the image processing system 120 or the user device 140 for subsequent analysis. In other embodiments, the image acquisition may be performed externally—such as by a third-party system—and the resulting image file may be transmitted or uploaded to the system 100 for processing.

FIG. 1B illustrates an example medical claim form 150 that includes multiple types of content features relevant to the extraction process. In particular, the form 150 comprises a plurality of key/value pairs, such as the key/value pair 151 corresponding to “Policy Holder Name: John A. Smith.” Such key/value pairs represent discrete data fields in which a key (or label) is associated with a corresponding value, forming structured information suitable for extraction and downstream processing. In addition to key/value pairs, the form also includes additional textual features 152 that are not structured as pairs but may still carry meaningful semantic information. These may include procedural descriptions such as a header “Treatment Details,” which help inform classification or context. Furthermore, the form 150 includes structural features 153, such as section dividers, which organize the content visually and hierarchically. These structural cues may assist in segmenting the document into logical sections, thereby aiding the machine learning model in identifying contextually relevant fields during classification or extraction. In some implementations, the textual and structural features may also assist in identifying candidate key/value pairs that are not explicitly formatted as such. For example, based on the structural and textual cues around the “Authorization” section in FIG. 1B, a model may infer that a date of the “Signature of Provider” and the corresponding “2024-03-28” constitute a key/value pair, even though the format differs from the standard pairs.

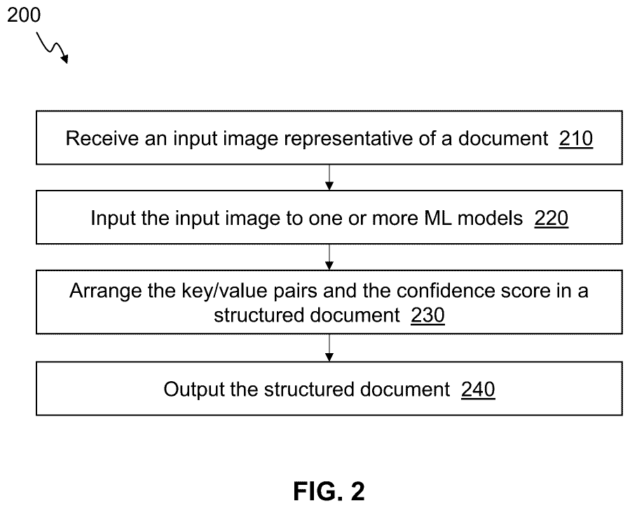

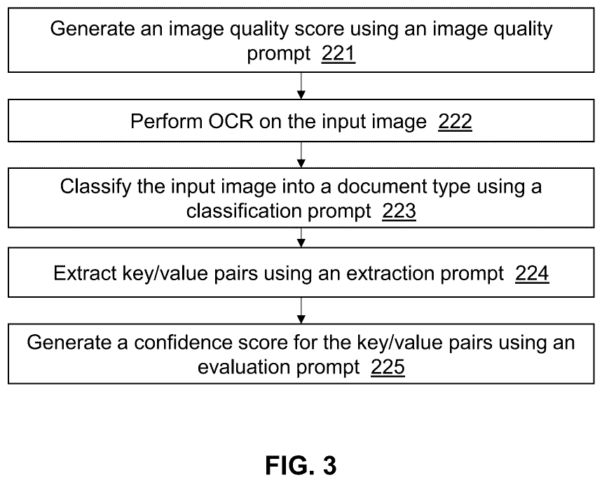

FIGS. 2 and 3 depict an example method 200 for interacting with a machine learning model, such as a multimodal LLM, according to some embodiments of the present disclosure.

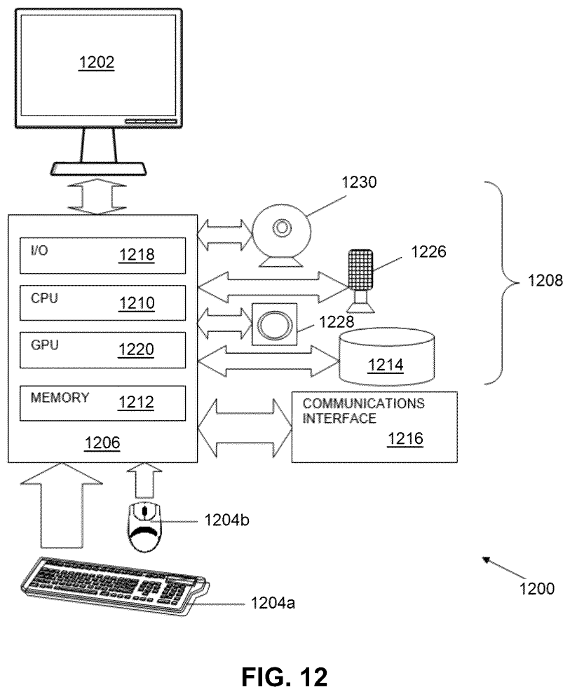

The method 200 may be performed by a system configured to process input images of documents, such as the system described with reference to FIG. 1A and FIG. 12, or any suitable computing device. In some embodiments, the method 200 is implemented using at least one processing unit (e.g., a central processing unit (CPU) 1210 or a graphics processing unit (GPU) 1220 of FIG. 12), which executes instructions stored in a memory, such as on a computer-readable medium (e.g., a memory 1212 of FIG. 12). For clarity, the operations of method 200 are described as being performed by the at least one processing unit; however, other components or devices may additionally or alternatively perform these operations. Although the operations of method 200 are depicted in a particular order, it is understood that they may be performed in a different order, omitted, and/or repeated without departing from the scope of the present disclosure.

At operation 210, the at least one processing unit receives an input image representative of a document. The input image may be provided by a user, retrieved from a pre-stored repository, or acquired from another source, such as a scanning device, a camera, or an external database. The input image depicts textual and structural features of the document, which may include content such as text, tables, checkboxes, or graphical elements. The textual features may include multiple key/value pairs of interest, such as the pair 151 shown in FIG. 1B, which are intended to be extracted during subsequent processing stages. In some embodiments, the input image may be displayed to the user through a GUI for review or confirmation.

At operation 220, the at least one processing unit inputs the received image to one or more machine learning models trained to perform various processing tasks. The one or more machine learning models are configured to execute a sequence of operations 221 to 225, as described below in relation to FIG. 3.

At operation 221, the one or more machine learning models generate an image quality score for the input image, indicating its suitability for Optical Character Recognition (OCR). The image quality score is generated using an image quality prompt that includes one or more image quality criteria, such as text legibility, image sharpness, contrast, noise level, and text alignment. In some embodiments, the image quality score provides an evaluation of whether the input image meets a predefined image quality threshold required for further processing.

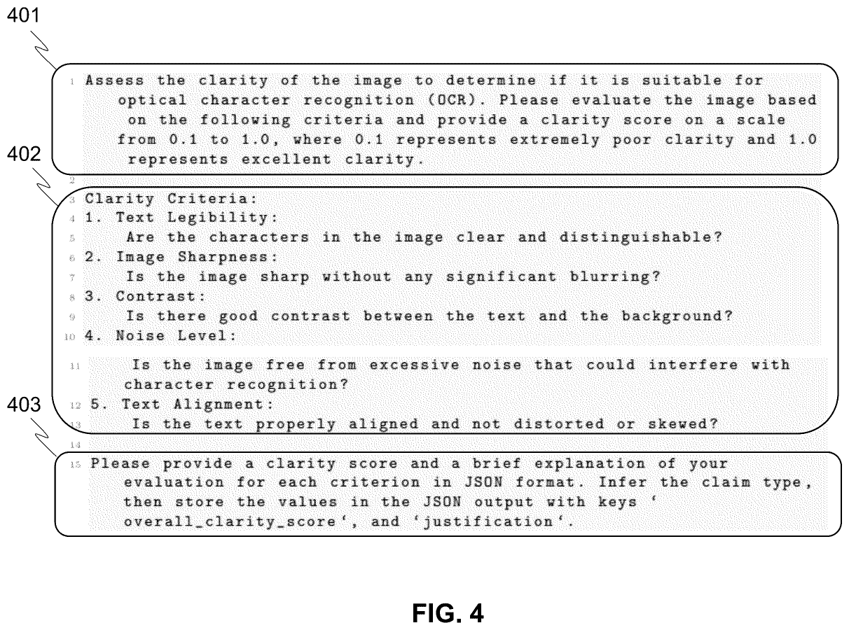

As a general principle, the image quality prompt may be generated using prompt engineering to ensure clarity and precision in guiding the LLM. An example image quality prompt is shown in FIG. 4. Specifically, the image quality prompt comprises one or more image quality criteria as well as definitions of the image quality criteria, such as text legibility and image sharpness, along with a natural language request to estimate the image quality on a numerical scale. These definitions and requests are configured to elicit meaningful responses from the LLM by framing the evaluation process in terms of predefined metrics and structured input. For instance, the prompt may request the LLM to assign scores based on predefined scales and justify these scores by referencing observed characteristics in the input image. By employing prompt engineering, the method leverages the LLM's capacity to process contextual information and generate structured output to derive the image quality score.

At operation 222, in response to the image quality score satisfying the threshold, OCR is performed on the input image to generate OCR output data. The OCR process may be carried out by one or more machine learning models configured for text detection and recognition or by a dedicated image processing system, such as image processing system 120 of FIG. 1A, which is integrated with the one or more machine learning models. This data includes textual features, such as extracted characters and words, as well as structural features, such as the layout and alignment of text regions within the document. The OCR output data provides the information for subsequent processing steps.

At operation 223, the one or more machine learning models classify the input image into a document type selected from a plurality of predetermined types. This classification is based on the textual and structural features obtained from the OCR output data and is performed using a classification prompt. For example, document types may include invoices, claim forms, or prescriptions. The classification step enables the system to identify the appropriate extraction rules or processing logic for the given document type.

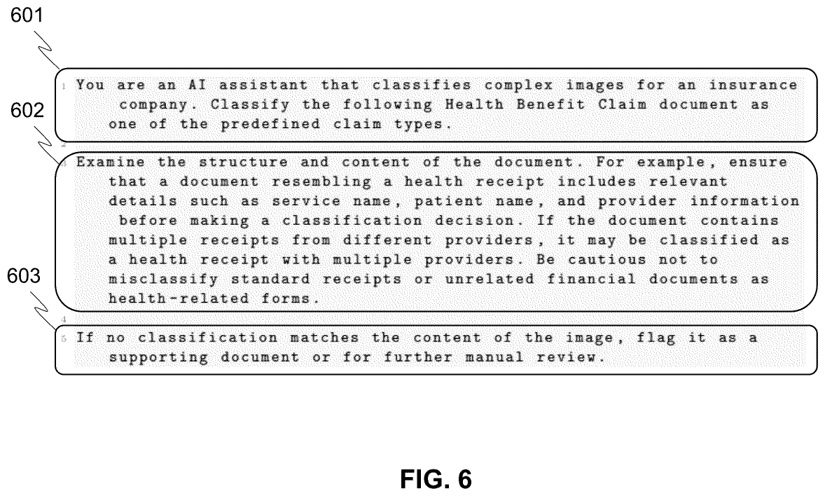

As a general principle, the classification prompt may be generated using prompt engineering to ensure precise and systematic guidance for the LLM. An example classification prompt is shown in FIG. 6. Specifically, the classification prompt includes the textual and structural features, such as keywords and layout elements, and specifies the plurality of predetermined types along with their corresponding classification criteria as well as definitions. Additionally, the classification prompt comprises a natural language request to classify the input image into one of the predetermined types. This configuration ensures that the LLM processes the classification prompt effectively by mapping the input features to the predefined categories. For example, the classification prompt may direct the LLM to analyze structural layouts, such as checkboxes or tables, and identify contextual information relevant to each document type, such as “Provider Details” or “Claim Amount”. This may include checking whether the visual formatting of the text matches typical patterns in the form layout. Deviations in alignment, spacing, or font may be indicators of lower consistency. By leveraging prompt engineering, consistent and accurate classification results can be achieved.

At operation 224, the one or more machine learning models extract one or more key/value pairs from the OCR output data using an extraction prompt including an extraction rule determined by the classified document type. For example, in the case of an invoice, the extraction rule may identify key fields such as “Invoice Number” or “Total Amount” and their corresponding values within the OCR output data. The extracted key/value pairs are organized for subsequent evaluation.

At operation 225, the one or more machine learning models generate a confidence score for the extracted key/value pairs. The confidence score is determined using an evaluation prompt that incorporates one or more textual evaluation criteria. These criteria may include visual consistency, which evaluates the alignment between the extracted key/value pairs and their corresponding regions in the input image. The confidence score provides a measure of the reliability of the extracted data and may be used to guide further processing or flag low-confidence data for manual review.

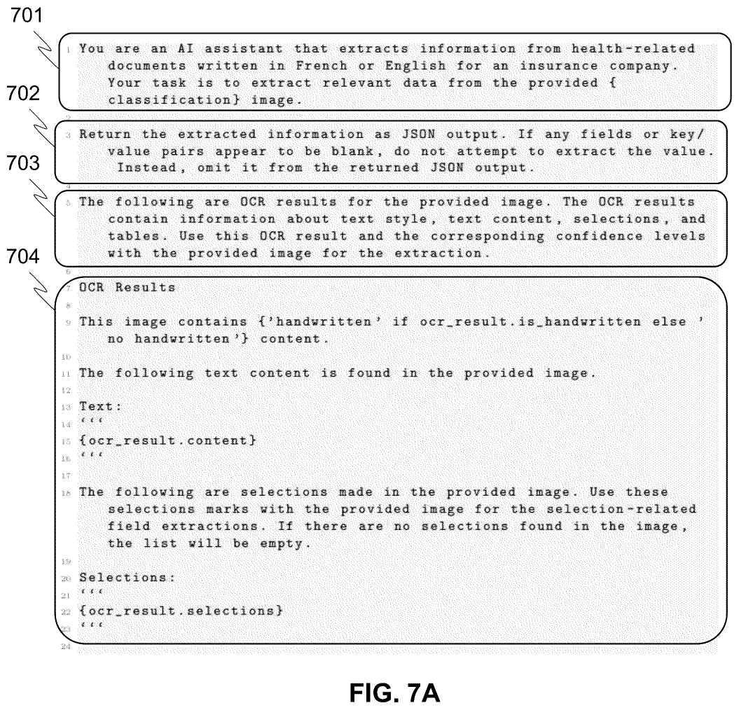

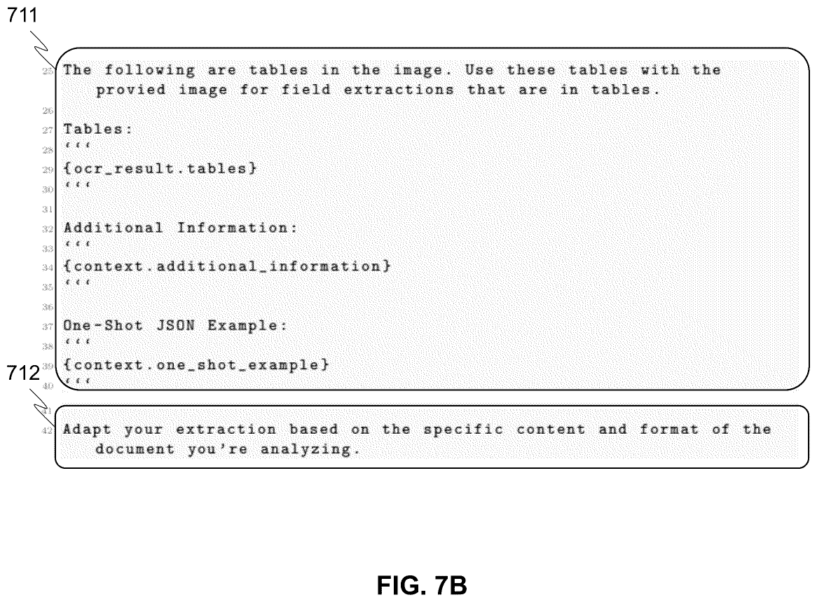

As a general principle, the evaluation prompt may be generated using prompt engineering to guide the LLM systematically in evaluating the extracted key/value pairs. An example evaluation prompt is shown in FIGS. 7A and 7B. The evaluation prompt comprises the extracted key/value pairs, one or more textual evaluation criteria as well as definitions of the textual evaluation criteria such as visual clarity, visual consistency, and relevance, and a natural language request to assign a confidence score for each key/value pair on a numerical scale. This configuration ensures that the LLM processes the evaluation input data by combining information from the input image, such as the regions containing the key/value pairs, with predefined evaluation criteria. For example, the evaluation prompt may request the LLM to assign a score for visual consistency based on whether the extracted key/value pair aligns with its corresponding region in the image and justify the score using positional or pixel-level comparisons. The LLM may be applied in a judging role, referred to herein as “LLM-as-Judge”, which involves using the LLM to evaluate extracted outputs against defined criteria and assign confidence scores or justifications in natural language. By leveraging prompt engineering, the evaluation process maintains a consistent standard across various input data.

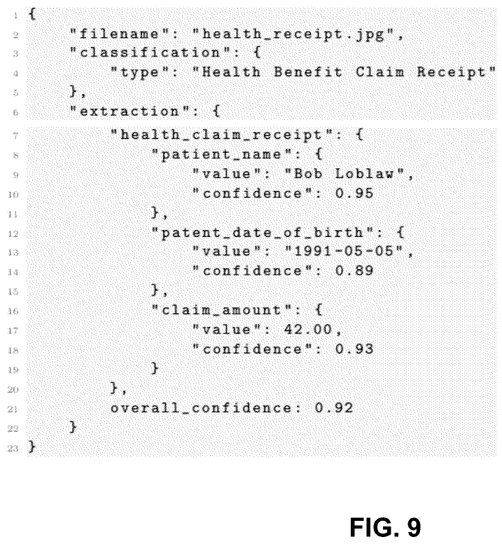

Returning to FIG. 2, at operation 230, the at least one processing unit arranges the extracted key/value pairs and the associated confidence score into a structured document. The structured document may be formatted according to predefined standards or templates suitable for downstream applications or integration with other systems. An example structured document is shown in FIG. 9.

At operation 240, the structured document is output by the at least one processing unit. In some embodiments, additionally, the structured document may be displayed to the user via a GUI, transmitted to an external system for further processing, or stored in a database for record-keeping or analytics. In some other embodiments, the output document may include annotations or metadata reflecting the confidence scores and other processing details.

The method 200 described above provides an efficient and structured approach for processing input images of documents, facilitating the extraction of data elements of interest with associated confidence scores. The described operations may be implemented using various machine learning architectures and prompt engineering techniques, as appropriate for the specific application and requirements of the system.

Prompt engineering in the framework described in the method 200 involves designing specific prompts that guide the machine learning models to perform their tasks effectively. These prompts are configured to align with the objectives of each stage of document processing, such as image quality evaluation, classification, extraction, and extraction evaluation. Once configured, the prompts can be pre-stored in a system memory (e.g., the memory 1212 of FIG. 12) or database (e.g., a storage 1214 of FIG. 12) for reuse, enabling consistent application across different instances of processing. FIGS. 4, 6, 7A, and 7B illustrate example prompts used in the embodiments described herein, which will be explained in detail in the following.

The prompts can be designed as templates with fixed language components that define the structure and rules (or instructions) for the models. Variables, such as dynamically generated content like extracted key/value pairs or document-specific attributes, can be incorporated into these templates at runtime. For example, an image quality prompt template may include fixed rules for assessing sharpness, contrast, and alignment, while the specific thresholds or criteria for these attributes are added based on the input image being processed. Similarly, a classification prompt template may include predefined language for identifying document types, with variables populated based on the textual and structural features extracted from the input image.

By pre-storing the templates, the system ensures that the prompts are consistently and efficiently applied during processing. These templates can be managed and retrieved based on the requirements of the processing stage. For example, during the extraction evaluation phase, the system can retrieve an evaluation prompt template configured with criteria, and dynamically insert variables corresponding to the extracted key/value pairs. This approach allows the system to maintain flexibility while ensuring that the prompts are applied in a structured and repeatable manner.

The prompts and images can be input into the one or more machine learning models in a structured manner to provide the models with the necessary information for processing. In some embodiments, the input data is generated in a form suitable for application to the machine learning models, so that the models can interpret and respond to the requests defined by the prompts. This process may involve tokenization, embedding, and concatenation of the textual and image-based inputs.

For textual prompts, the at least one processing unit can tokenize the text into discrete parts, such as words or phrases, which can then be embedded into a vector space. The tokenization may transform the textual content into a format that the machine learning models can interpret. For images, the tokenization process may involve segmenting the image into patches, regions of interest (Rol), or other suitable tokenization formats that capture the spatial and visual information of the image.

Once the prompts and images are tokenized, the at least one processing unit can concatenate the tokenized textual and image inputs into a unified sequence. This concatenated sequence serves as the combined input for embedding into a vector space, ensuring that the machine learning models can process both modalities simultaneously. The embedding process can involve one or more techniques, such as convolutional neural networks (CNNs), linear projections, learned embeddings, or graph neural networks (GNNs), to convert the concatenated tokens into a numerical representation suitable for model input.

### 1. Four-Stage Framework

The following example illustrates an embodiment of the four-stage framework for processing health benefit claims. The framework leverages a structured and systematic approach to extract key/value pairs from document images, encompassing an Image Quality Evaluation stage to assess the suitability of the input image for processing, an Image Classification stage to determine the document type, an Image Extraction stage to retrieve relevant information based on the document type, and an Extraction Evaluation stage to assess the accuracy and reliability of the extracted data. In one embodiment, the four stages may utilize a multimodal LLM. While it is possible for a single LLM to perform all stages and tasks within the framework of the embodiments described herein, in some implementations, different classes of models may be used for distinct functional purposes. For example, the stages of quality assessment, classification, and extraction may be performed using a first model class tuned for structured output generation, while the evaluation stage may employ a second model class tuned for interpretability and scoring based on natural language reasoning. This division of model responsibilities may improve performance and flexibility across heterogeneous processing tasks. Each stage contributes to a comprehensive and accurate data processing methodology, as described in detail below.

### 1.1. Image Quality Evaluation

In the first stage, referred to as the Image Quality Evaluation stage, the input image undergoes an assessment to determine its suitability for OCR. This assessment is based on predefined criteria designed to evaluate various attributes of the image that directly impact the accuracy of OCR. These criteria include, but are not limited to, one or more of text legibility, image sharpness, contrast, noise level, or text alignment. These factors can ensure that the image satisfies the quality requirements necessary for accurate OCR processing.

The evaluation process is guided by a predefined image quality prompt, an example of which is illustrated in FIG. 4. The image quality prompt directs the LLM to assess various attributes of the input image and provide an image quality score alongside a justification for each evaluation criterion. The image quality prompt may be structured into multiple logical blocks, each corresponding to a specific function.

FIG. 4 illustrates a detailed example of the predefined image quality prompt used in the image quality evaluation process. The image quality prompt provides specific rules for analyzing key attributes of the input image, and it may specify one or more image quality criteria to guide the analysis. For example, it may require the LLM to evaluate whether the characters in the image are clear and distinguishable, whether the image exhibits significant blurring, and whether the text is properly aligned and free of distortion. These targeted queries align with predefined criteria to facilitate the image quality evaluation. As shown in FIG. 4, block 401 of the prompt functions as a role definition and task introduction block. It introduces the task to the LLM, instructing it to assume the role of an evaluator tasked with assessing the clarity of an image for OCR suitability, and outlines the context for scoring.

The image quality prompt outlines a structured set of rules for the LLM to evaluate clarity criteria, including text legibility, image sharpness, contrast, noise level, and text alignment. Each criterion is accompanied by specific questions, such as “Are the characters in the image clear and distinguishable?” or “Is the image sharp without any significant blurring?” These rules are organized in block 402, which serves as a criteria definition block. This block explicitly enumerates the evaluation criteria, each accompanied by one or more guiding questions to help the LLM interpret the criteria consistently across different input images.

Additionally, the image quality prompt specifies that the evaluation results be formatted in JavaScript Object Notation (JSON), with fields like overall_clarity_score and justification to ensure interpretability. This specification is set out in block 403, which functions as the output formatting block. This block defines the expected structure and field names for the model's response, allowing downstream systems to parse and utilize the output. This structured approach ensures that each criterion is assessed methodically to allow the LLM to generate a comprehensive and actionable output. The example shown in FIG. 4 may serve as a reusable template, ensuring consistency in evaluations across a wide range of input images.

The image quality prompt is configured in a manner that clearly delineates the scope of each evaluation criterion. For example, for text legibility, the LLM may analyze the clarity and distinctiveness of individual characters. This evaluation may involve comparing the pixel-level features of the characters to a set of predefined patterns or using heuristic rules embedded in the LLM to recognize text clarity. For image sharpness, the LLM may assess the gradients or edge transitions within the image, identifying any regions that exhibit blurring. For text alignment, the LLM may evaluate the relative positioning of text lines or blocks to determine if the text is skewed or distorted beyond acceptable limits.

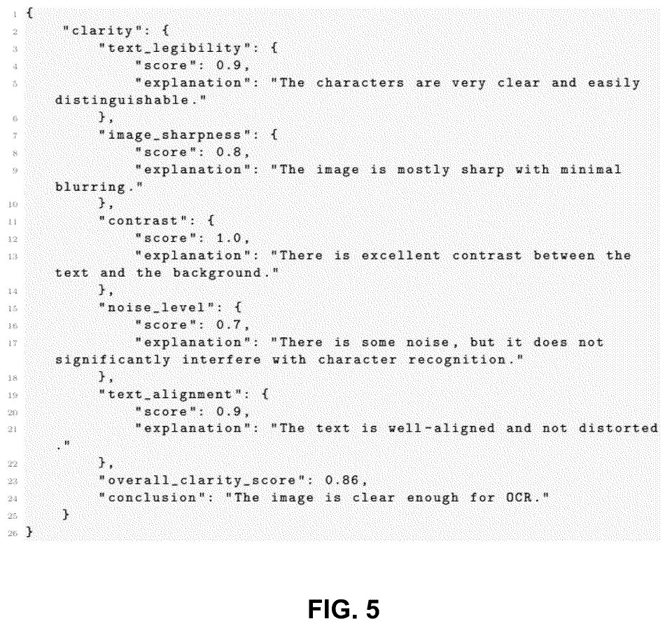

Each criterion may be scored individually on a scale from 0.1 to 1.0, with higher scores indicating better quality (while in some other examples, lower scores may indicate better quality). The overall clarity score may be calculated to be an average of these individual scores, as shown in FIG. 5. This overall clarity score may be accompanied by a justification for each criterion, structured in the JSON format, for example. The JSON output provides a clear and interpretable explanation of the LLM's evaluation to enable downstream systems to take appropriate actions based on the clarity score. Other data formats, such as Extensive Markup Language (XML) or Yet Another Multicolumn Layout (YAML), may be alternatively used to structure the image quality evaluation output, depending on the requirements of the downstream systems.

FIG. 5 provides an example of the structured output generated during the Image Quality Evaluation stage. The JSON output includes detailed assessments of each criterion: text_legibility, image_sharpness, contrast, noise_level, and text_alignment. Each criterion is assigned a numerical score along with an explanation justifying the score. For example, the text_legibility criterion is scored 0.9, with an explanation stating, “The characters are very clear and easily distinguishable.” Similarly, contrast receives the highest score of 1.0, supported by the observation that “There is excellent contrast between the text and the background.” The overall clarity score, calculated as an average of these individual scores, is reported as 0.86, accompanied by a conclusion: “The image is clear enough for OCR.” The conclusion can be drawn by comparing with a predetermined threshold, which will be described in the following.

The structured approach, as shown in FIG. 5, ensures transparency in the evaluation process and provides actionable insights. The inclusion of both scores and explanations allows downstream systems to identify specific areas where quality issues may arise and determine appropriate preprocessing steps. For example, if noise_level scores low, targeted noise reduction techniques may be applied to enhance the image quality. The JSON structure may also facilitate integration with automated workflows, so that the evaluation results can be readily consumed by subsequent stages in the OCR framework. It should be understood that the evaluation criteria may be selectively chosen based on their relevance to OCR accuracy or according to the user's preference.

In some embodiments, if the image quality score satisfies a predefined threshold, the input image proceeds to the next stage of the framework. However, if the score falls below the threshold, the image may be flagged for preprocessing. The preprocessing, such as cropping, rotating, resizing, or enhancing contrast, may then be applied to improve the image quality such that the text can become clearer or more recognizable. After preprocessing, the image may be re-evaluated to determine whether its image quality score has improved sufficiently for further processing. In some embodiments, such preprocessing may be applied to each image by default, prior to determining the image quality score and the initial quality assessment; the resulting image quality score may then be used to determine whether further processing should be carried out. In some embodiments, the preprocessing may be performed by an image processing system, such as the image processing system 120 shown in FIG. 1A, which may apply the processing automatically or in response to system prompts. It should be understood that the preprocessing may be performed regardless of the determination of the image quality score.

### 1.2. Image Classification

After the image is determined to be suitable for OCR, it is classified into one of several predefined document types. The classification allows that subsequent extraction processes to be tailored to the specific characteristics of the document. Document types may include, for example, invoices, claim forms, prescriptions, and other health-related documents, as defined by the system's configuration or business requirements. In addition to medical-related documents, image classification can also be applied to documents in other fields, such as inventory lists, purchase orders or quotations, which are widely available in general business, depending on the requirements or application area.

The classification process is guided by a predefined classification prompt, as illustrated in FIG. 6, which instructs the LLM to analyze the structure and content of the document. The classification prompt directs the LLM's attention to relevant features such as textual elements, structural layouts, and context-specific patterns. As shown in block 601 of FIG. 6, the classification prompt begins with a task definition block that establishes the model's functional role, in this case, acting as an assistant responsible for document classification. For example, the classification prompt may instruct the model to confirm the presence of details like “Service Name”, “Patient Name”, and “Provider Information” in health-related documents before making a classification decision. Block 602 contains the classification rule logic, which outlines specific structural and contextual indicators the model should consider when making a classification decision. For complex cases, such as documents with multiple receipts from different providers, the classification prompt may specify that these should be categorized as health receipts with multiple providers. If the document does not match any predefined type, it may be flagged for manual review or categorized as a supporting document. This fallback logic is expressed in block 603, which functions as the exception handling portion of the prompt.

FIG. 6 demonstrates how the classification prompt ensures categorization by guiding the LLM through structured criteria and fallback procedures. The structured rules minimize misclassification errors, so that the model can identify relevant document types reliably. This process ensures that only appropriately classified documents proceed to subsequent stages of processing.

Classification criteria focus on attributes that distinguish document types. For example, health receipts may include fields such as “Service Name”, “Patient Name”, and “Provider Information”, while invoices may feature itemized costs and payment details, and claim forms may include “Claim Amount” and “Service Date”. These attributes may be identified from patterns extracted from the OCR output. The classification rule logic in block 602 may explicitly reference these criteria to help the LLM differentiate between document categories.

The LLM determines the document type by analyzing textual and structural features, such as keywords and layout elements. Some examples of the layout elements may include checkboxes, tables, headers, angle of rotation, and section breaks. It compares these features with predefined patterns for each document type. In some embodiments, the model may combine rule-based systems, statistical methods, and trained neural networks to improve classification accuracy. The rule logic block may also implicitly support hybrid evaluation methods by providing open-ended interpretation of content structure and context.

If the LLM encounters a document that does not match any predefined category, it may flag the document for further review. As illustrated in FIG. 6, the classification prompt may ask the LLM to categorize such documents as supporting documents or suggest a manual review. This functionality is outlined in block 603, which provides procedural handling for classification uncertainty.

The classification prompt may be pre-configured as a reusable template, allowing for consistent application across different images. The classification prompt defines the scope of analysis, specifying attributes such as keywords, field arrangements, and contextual information to be considered during classification. By defining explicit task roles, targeted evaluation criteria, and fallback procedures in blocks 601, 602, and 603, respectively, the classification prompt achieves modularity and clarity in prompt design. For example, the prompt may direct the LLM to confirm the presence of “Provider Details” or “Service Data” in documents resembling invoices or to avoid misclassifying unrelated financial documents as health-related forms.

The classification stage ensures that relevant extraction rules are applied during the next stage. By tailoring the analysis to the identified document type, the system can better align with domain-specific requirements to improve the overall quality and reliability of the processed data.

### 1.3. Image Extraction

After the document type is classified, the system extracts specific key/value pairs from the OCR output using predefined extraction rules tailored to the classified document type. These rules specify the fields relevant to the document type to ensure that the extraction is contextually appropriate.

The extraction process is guided by extraction prompts such as the ones illustrated in FIGS. 7A and 7B. These extraction prompts provide explicit rules for identifying and retrieving relevant data. The extraction prompts may be configured to be adaptable to various document formats to accommodate differences in layout, language, and content.

FIG. 7A illustrates the rules provided to the system for handling OCR results and selection marks. As illustrated in block 701 of FIG. 7A, the extraction prompt begins with a task specification, instructing the LLM to assume the role of an assistant responsible for extracting structured data from health-related claim documents. This block defines the scope of the extraction task and clarifies that the input will relate to a classified document type.

FIG. 7A further illustrates various extraction rules applied to handle OCR results and visual content in the image. Block 702 sets forth an output specification, directing the LLM to format the extraction results as structured JSON data and to omit fields with blank values. This instruction ensures output consistency and avoids generation of speculative or inferred values. Block 703 introduces a data context specification, providing the model with access to relevant OCR results and associated confidence levels. Block 704 defines the template structure for how to extract textual content, determine handwriting status, and extract region-based selections. It may also include extraction anchors such as labels (e.g., “Text:” and “Selections:”) and embedding fields for image-derived metadata. Together, these blocks define a structured and modular prompt, guiding the LLM in handling various visual and textual artifacts associated with the document.

The extraction rules may be determined based on the document type identified during the classification stage. For example, in the case of an invoice, the extraction rules may direct the LLM to identify itemized costs, total amounts, and payment details by analyzing table structures and labels. For a health receipt, the rules may focus on extracting service dates, patient names, and provider details. These rules ensure that fields of interest are retrieved.

In cases where fields are blank or absent, the system may follow the rules specified in the prompt to omit or remove those fields from the output. As described in block 702 of FIG. 7A, the LLM is explicitly directed to omit any key/value pairs that include blank values from the final JSON output. For example, as shown in FIG. 7A, if a key/value pair includes a blank value, the prompt directs the system not to attempt inferring or filling in the missing data. This approach prevents the generation of inaccurate or speculative information, ensuring that the output remains reliable and precise.

FIG. 7B expands upon block 704 of the extraction prompt shown in FIG. 7A by introducing additional prompt blocks that enhance the structured extraction process. Specifically, block 711 provides table-handling guidance to the large language model (LLM). It instructs the LLM to use any tables identified in the OCR output (e.g., ocr_result.tables) in conjunction with the image when performing field extractions involving tabular data. This rule is particularly relevant for document types such as invoices or service records, where relevant values such as item descriptions, quantities, and costs may appear within a table. Block 711 also comprises a section of the prompt that provides pre-filled structured fields for the model's reference. These include contextual metadata (context.additional_information) and a one-shot JSON example (context.one_shot_example). This is the portion of the prompt that provides the LLM with specific examples and auxiliary information to guide the formatting and structure of the expected output. These inputs serve as anchors for consistency and help align the model's outputs with predefined expectations across various document types. Block 712 concludes the prompt with a general rule to the model. This is the portion of the prompt that instructs the LLM to adapt its extraction behavior based on the specific content and format of the document under analysis.

The extraction process may also involve preprocessing operations applied to the input image, such as cropping, rotating, or resizing, to better align the content with expected text regions. For example, if the OCR engine outputs metadata indicating an image is skewed or rotated, the system may invoke image alignment techniques to correct the document orientation. This may improve the ability of the LLM to match extracted features with their original spatial locations. Preprocessing may be handled by a component such as the image processing system 120, which may apply these enhancements prior to or during extraction.

The final structured output of the extraction process may be represented in a machine-readable format such as JSON. As shown in FIG. 7B, the extraction prompt provides formatting references and exemplar data to ensure the consistency of output. This structured output enables downstream systems to reliably interpret the extracted key/value pairs for further processing. In other embodiments, alternate data formats such as Extensible Markup Language (XML) or YAML Ain′t Markup Language (YAML) may be used, depending on the compatibility and integration requirements of the target systems. Structured formatting facilitates automated handling of the extracted data for workflows such as health claim adjudication, reporting, or auditing.

### 1.4. Extraction Evaluation

In the fourth stage, the extracted key/value pairs undergo an extraction evaluation to assess accuracy and reliability. This stage employs an evaluation prompt configured to guide the LLM in applying textual evaluation criteria to the extracted data. The evaluation in this example focuses on three criteria: visual clarity, visual consistency, and relevance. The LLM may be applied in a judging role, referred to herein as “LLM-as-Judge”, to assess each extracted key/value pair with respect to these criteria and assign confidence scores accordingly.

Visual clarity, which may be weighted at 25%, examines whether the text in the image is clear and readable. In some embodiments, visual clarity may be determined from the image quality evaluation, such as the image quality score, in relation to the part of the input image containing the key/value pairs of interest.

Visual consistency, which may be weighted at 50%, assesses whether the extracted key/value pairs align closely with their corresponding regions in the input image. For example, if the value extracted for “Invoice Total” matches the position and content of the total displayed in the image, it demonstrates a high degree of visual consistency. This criterion may involve comparing pixel-level features of the image with the extracted text to assess positional and content alignment.

Relevance, which may be weighted at 25%, evaluates whether the extracted data is contextually appropriate for the classified document type. The relevance is determined by assessing whether the extracted key/value pairs correspond to fields expected for the classified document type based on a predefined rule. For example, if the document type is classified as a health receipt, fields such as “Service Name” or “Provider Name” are considered relevant because they align with the expected fields for this document type, while unrelated data such as financial details would be deemed irrelevant. This criterion ensures that the extracted key/value pairs are meaningful and appropriate in the context of the document's purpose.

The evaluation prompt may be configured to encapsulate these criteria in a structured query that the LLM can process. For example, the evaluation prompt may specify rules such as: “Compare the extracted key/value pair with its corresponding region in the image for alignment and accuracy. Assign a score based on visual consistency and visual clarity. Evaluate the relevance of the key/value pair to the document type.” This structured approach guides the LLM-as-judge in performing a consistent and explainable evaluation.

It should be understood that the textual evaluation criteria may include only one criterion, such as visual consistency, while visual clarity and relevance are optional. However, the textual evaluation criteria may include other combinations of criteria or an additional criterion. It should also be understood that weights associated with the criteria can be varied.

Each key/value pair may be assigned a confidence score ranging from 0.0 to 1.0 based on the weighted average of the three criteria. For example, if a key/value pair for “Claim Amount” visually aligns with the text in the image, a high confidence score such as 0.95 may be assigned for visual clarity. However, if a mismatch or ambiguity is detected, the confidence score may be lower, prompting the system to flag the field for review or exclude it from the final output.

The individual confidence scores may be aggregated and used to calculate an average confidence level for the entire document, according to the weights assigned for the criteria. This provides an overall measure of the extraction quality, enabling downstream systems to make informed decisions about whether additional manual review is needed.

The evaluated key/value pairs, along with their confidence scores and, optionally, justification text generated by the LLM, may be arranged into a structured document. This document may be formatted using predefined templates that include metadata such as confidence levels and evaluation justifications. For example, annotations may indicate that a particular field was flagged for low confidence, aiding in manual review or further processing.

The resulting structured document, which includes the extracted key/value pairs and confidence score(s) and any justification text from the LLM, may be output in a machine-readable format such as JSON, XML, or YAML. This document can then be integrated into downstream workflows to enable processing for applications such as claims analysis, invoicing, or reporting.

### 2. Evaluation Methodology

As a complement to the fourth stage described above, a more comprehensive evaluation methodology can be implemented in order to assess the accuracy and reliability of the extracted key/value pairs. This methodology may incorporate a multi-faceted approach leveraging heuristic evaluators, human evaluators, and/or a “LLM-as-Judge” framework. Each evaluator type can serve a distinct and complementary role in ensuring robust evaluation while balancing scalability and precision. However, it should be understood that the evaluation may involve only one evaluator, such as the LLM-as-Judge evaluator.

### 2.1. Heuristic Evaluators

Heuristic evaluators utilize predefined rules and automated tools to evaluate the structural integrity and format consistency of the extracted key/value pairs. This evaluation method acts as the first line of defense, identifying obvious errors without requiring human intervention.

Predefined rules are criteria established to enforce specific standards in the extracted data. These rules may include conditions such as data type validation, length constraints, format requirements, and schema compliance. For instance, a predefined rule may verify that a “date of birth” field follows the “YYYY-MM-DD” format or that a “claim amount” field contains a numeric value within a valid range. Such rules assist in identifying deviations or errors in the data that may otherwise go undetected in the absence of human review.

Automated tools used in heuristic evaluation leverage these predefined rules to process the extracted key/value pairs efficiently and ensure compliance with the desired criteria. An example of automated tools is Pydantic library, which may be effective because it not only validates data types and formats but also enforces structural consistency through the use of predefined schemas.

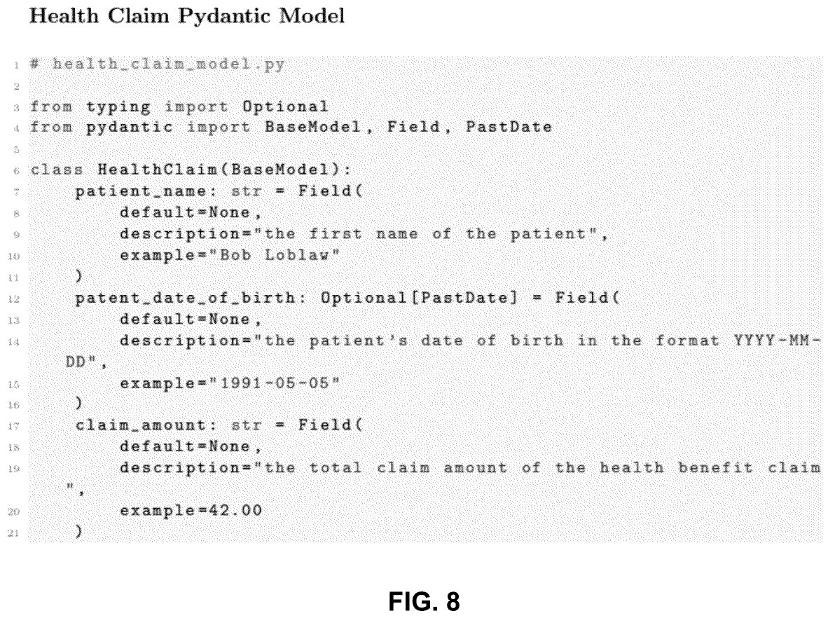

FIG. 8 illustrates an example implementation of a Pydantic model employed to guide the evaluation process and validate the structural correctness of the extracted outputs. The model, named Health Claim, defines a schema for health claim data with fields such as patient_name, patient_date_of_birth, and claim_amount. Each field includes attributes specifying default values, descriptions, and examples to ensure clarity and compliance.

For example, the patient_name field is defined as a string with a default value of None, a description indicating that it represents “the first name of the patient”, and an example value of “Bob Loblaw”. Similarly, the patient_date_of_birth field, marked as optional, enforces the format “YYYY-MM-DD” and includes a sample date, “1991-05-05”, to demonstrate compliance with the rule. The claim_amount field ensures that the extracted value is numeric, with an example value of 42.00 provided for reference.

The Pydantic model ensures that extracted data conforming to this schema is structurally sound and ready for downstream processing. During evaluation, if a key/value pair does not align with the specified rules (e.g., a date in an invalid format or a missing mandatory field), the heuristic evaluator flags the discrepancy for review or correction. Heuristic evaluators, including implementations like the one shown in FIG. 8, provide a cost-effective and rapid method for filtering out inaccuracies.

### 2.2. Human Evaluators

Human evaluators assist in delivering nuanced, qualitative assessments of the extracted data. These evaluators are typically trained personnel with domain-specific knowledge that enables them to evaluate the extracted outputs according to one or more criteria.

For example, in the evaluation process, human reviewers can sample a subset of the extracted data and assign scores based on visual clarity, visual consistency, and relevance. This qualitative input serves as a benchmark for validating the decisions made by both heuristic evaluators and the LLM-as-Judge. Furthermore, human evaluators can address cases that require contextual understanding or industry-specific expertise.

### 2.3. LLM-as-Judge Evaluators

The LLM-as-Judge evaluator represents an automated evaluation mechanism that integrates human-like grading criteria into its assessment process. This system leverages predefined prompts to evaluate key/value pairs extracted from the input data against criteria such as visual clarity, visual consistency, and relevance, as discussed above in relation to Stage 4 of the four-stage framework. By applying these criteria, the LLM-as-Judge provides a scalable and efficient solution for processing a large volume of extracted data while maintaining high evaluation standards.

Compared with heuristic evaluators, which focus primarily on structural validation and compliance with predefined rules, the LLM-as-Judge can perform a more sophisticated analysis. It assesses key/value pairs by comparing the extracted output against expected results derived from the contextual understanding of the document. This evaluation strikes a balance between automated rule-based evaluation and manual human judgment.

FIG. 9 illustrates an example implementation of the LLM-as-Judge evaluation process, where confidence scores are assigned at both the field level and the overall document level. In this example, the input document, labeled “health_receipt.jpg”, has been classified as a “Health Benefit Claim Receipt”. The classification stage ensures that the document type is correctly identified, which informs the contextual relevance criteria used during evaluation.

The “extraction” section of FIG. 9 shows the key/value pairs extracted from the document, along with their respective confidence scores. For example:
(a) The field patient_name contains the value “Bob Loblaw”, with a confidence score of 0.95, indicating high confidence of accuracy.
(b) The field patient_date_of_birth contains the value “1991-05-05”, with a confidence score of 0.89, reflecting slightly dropped confidence compared with the patient_name field.
(c) The field claim_amount contains the value 42.00, with a confidence score of 0.93, indicating high confidence of accuracy.

An overall confidence score of 0.92 is assigned to the entire document. This score is calculated as a weighted average of the individual field-level confidence scores (as discussed in relation to Stage 4 of the four-stage framework), reflecting the overall reliability and quality of the extraction process. The high overall confidence score in this example suggests that the extracted key/value pairs collectively meet the established evaluation criteria.

The structured output generated by the LLM-as-Judge evaluator, as depicted in FIG. 9, includes both the extracted key/value pairs and their associated confidence scores. This output can be formatted in JSON, XML, or other machine-readable formats to facilitate seamless integration with downstream systems. Additionally, the confidence scores provide actionable insights for further processing. For example, fields with lower confidence scores may be flagged for manual review, so that any ambiguities or inconsistencies are resolved before the data is used in subsequent applications, such as claims analysis or reporting.

### 2.4. Statistical Analysis

To ensure the reliability of the evaluation process, statistical methodologies may be employed. These include confidence interval estimation for evaluating the accuracy of key/value pair extraction and inter-rater reliability measures, such as the F1 score, to assess agreement between human evaluators and the LLM-as-Judge.

Confidence intervals provide a quantitative measure of the extraction accuracy, enabling the system to identify the margin of error and improve precision. Meanwhile, inter-rater reliability metrics validate the alignment of human and LLM-based evaluations, ensuring consistency across different evaluative frameworks.

The integration of one or more of these evaluation methodologies provides a robust mechanism for assessing and improving the quality of key/value pair extractions, so that extracted data meets the accuracy and relevance standards required for downstream applications.

### 3. Pre-Production Scenario

A practical example was applied to the extraction processes on a dataset of 9,330 paper health claim images. This pre-production phase aimed to evaluate the effectiveness of the vision-enabled model and the prompt engineering strategies before full-scale deployment. By conducting this evaluation, the system's accuracy and reliability standards were assessed to ensure operational readiness.

### 3.1. Confidence Interval Analysis

To establish statistical significance for the evaluation, a representative sample size was determined. For an infinite population, the required sample size (no) was calculated using the formula:

$${n}_{0} = \frac{{Z}^{2} \cdot p \cdot ( 1 - p )}{{E}^{2}} \qquad ( 1 )$$

where Z=1.96, representing a 95% confidence level; p=0.5, a conservative estimate for the population proportion; and E=0.05, corresponding to a 5% margin of error.
Substituting these values yielded:

$${n}_{0} = \frac{( 1.9 {6}^{2} ) \cdot 0.5 \cdot ( 1 - 0.5 )}{0.0 {5}^{2}} = \frac{3.8 \; 416 \cdot 0.25}{0.0025} = \frac{0.9604}{00025} \approx 384.16 \qquad ( 2 )$$

Since the population size (N) is finite, the sample size was adjusted using the formula:

$$n = \frac{N \cdot {n}_{0}}{N + {n}_{0} - 1} \qquad ( 3 )$$

Substituting N=9,330 and n₀=384.16:

$$n = \frac{9 \; 330 \cdot 38 \; 4.16}{9330 + 384.16 - 1} = \frac{3584500.8}{9713.16} \approx 369 \qquad ( 4 )$$

Thus, the statistically significant sample size for the dataset was approximately 369.

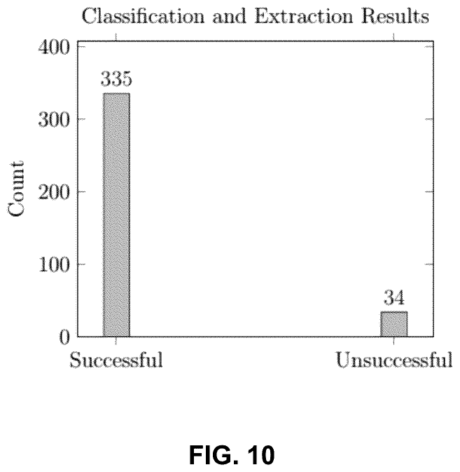

For the sample data, 335 out of 369 classifications and extractions were successful, as also shown in FIG. 10, in which the distribution of classification and extraction results are illustrated, showing 335 successful outcomes and 34 unsuccessful ones. As a result, it yielded a sample proportion (p) of approximately 0.9084:

$$\hat{p} = \frac{335}{369} \approx 0.9084 \qquad ( 5 )$$

The standard error (SE) was calculated as:

$$SE = \sqrt{\frac{\hat{p} \cdot ( 1 - \hat{p} )}{n}} = \sqrt{\frac{0.9 \; 084 \cdot ( 1 - 0.9084 )}{369}} = \sqrt{\frac{0.0832}{369}} \approx \sqrt{0.0002254} \approx 0.015 \qquad ( 6 )$$

Using a 95% confidence level (Z=1.96), the margin of error (ME) was determined as:

$$M E = Z \cdot SE = 1 \text{.96} \cdot 0.015 \approx 0.0294 \qquad ( 7 )$$

The confidence interval was then calculated as:

$$Confidential Interval = \hat{p} \pm M E = 0.9084 \pm 0.0294 \qquad ( 8 )$$

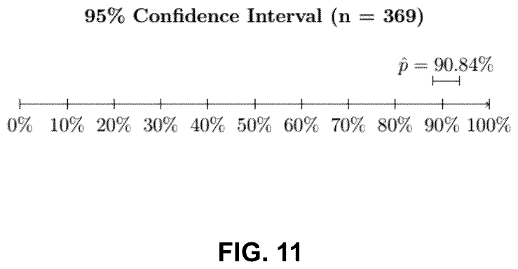

Thus, the 95% confidence interval for the proportion of successful classifications field extractions was approximately (0.8790, 0.9378), as shown in FIG. 11, which presents the calculated confidence interval for the sample, emphasizing the range of success rates expected across the entire dataset.

This confidence interval indicated that, with 95% confidence, the true proportion of successful classifications and extractions lied between 87.90% and 93.78% for the entire population of 9,330 documents. This range provided a reliable benchmark for evaluating the system's performance during the pre-production phase and offered a quantitative measure of its effectiveness.

In practical terms, the lower bound of the confidence interval (87.90%) suggested that at least 87.90% of the classifications and extractions were expected to be successful. This represented a conservative estimate, ensuring that the system meets minimum reliability expectations. Conversely, the upper bound (93.78%) indicated that up to 93.78% of the classifications and extractions could be successful under optimal conditions. The interval thus accounted for variations in performance due to factors such as document complexity, layout diversity, and OCR quality.

This confidence interval analysis served as an essential diagnostic tool for assessing the system's readiness for deployment. By providing a clear range of expected success rates, it could be determined whether the system met the accuracy requirements for its intended use. Additionally, the confidence interval offered actionable insights, such as identifying scenarios where preprocessing or additional refinements may further improve performance.

### 3.2. Evaluation of the LLM-as-Judge Using F1 Score

To evaluate the LLM-as-Judge approach, a sample of 369 documents, previously annotated by human evaluators, was analyzed. Of these, the LLM's evaluation matched the human annotations in 350 instances, resulting in an observed agreement rate of 95%.

While accuracy was a useful metric, it sometimes did not fully account for cases of class imbalance. To address this, the F1 score was employed as a comprehensive measure that considers both precision and recall. Precision represented the proportion of correctly identified positive instances out of all instances classified as positive by the LLM, and it was defined as:

$$Precision = \frac{True Positives}{True Positives + False Positives} \qquad ( 9 )$$

and recall referred to the proportion of correctly identified positive instances out of all actual positive instances, and it was defined as:

$$Recall = \frac{True Positives}{True Positives + False Negatives} \qquad ( 10 )$$

Using the sample data where True Positives (TP)=340, False Positives (FP)=10, and False Negatives (FN)=19, the precision and recall were calculated as:

$$Precision = \frac{340}{340 + 10} = \frac{340}{350} = 0 \text{.9714} \qquad ( 11 )$$

$$Recall = \frac{340}{340 + 19} = \frac{340}{359} = 0.947 \qquad ( 12 )$$

The F1 score was then computed as:

$$F 1 = \frac{2 \times Precision \times Recall}{Prec i s i o n + Recall} = \frac{2 \times 0.9714 \times 0.947}{0.9714 + 0.947} = \frac{1.8398}{19184} = 0.959 \qquad ( 13 )$$

The F1 score of 0.959 indicated a balanced performance by the LLM in terms of both minimizing false positives and false negatives. This metric provided a robust evaluation of the LLM's reliability and its potential to replicate human evaluation accuracy in document processing tasks.

The use of the F1 score provided a robust indication of the LLM-as-Judge approach's reliability, ensuring that its performance was not only accurate but also balanced in terms of precision and recall. This further demonstrated that LLMs served as effective tools for automating document evaluations, producing results comparable to those of human experts.

### 4. Post-Production Analysis

In the post-production stage, the focus may shift to ensuring the sustained performance of the key/value pair extraction system. Continuous monitoring and evaluation assist in maintaining system reliability, detecting performance degradation, and addressing shifts in data characteristics over time. This stage emphasizes the implementation of strategies that adapt the system to evolving operational requirements and input distributions. This section lists a few possibilities for the post-production analysis and it should be understood that additional analyses may be contemplated.

### 4.1. Real-Time Monitoring

Key/value pair extraction results for each image may be stored in a centralized database, which is integrated with visualization tools such as PowerBI™, Grafana™, or Tableau™. Real-time dashboards display performance metrics, enabling immediate detection of anomalies and facilitating prompt responses to potential issues. For example, if a sudden drop in extraction accuracy is observed, this may trigger an alert for further investigation, ensuring minimal disruption to system operations.

### 4.2. Continuous Sampling and Confidence Interval Evaluation

As with the pre-production analysis, a confidence interval may be constructed after a production deployment. Regular sampling of images may be performed periodically (e.g., weekly or monthly) to re-evaluate system performance. This approach not only helps identify any deviations from expected performance, but also helps build a statistically sound evaluation of the system over time. In addition, the periodic nature of sampling allows the system to account for potential changes in input distributions or operating conditions. By reassessing at regular intervals, the system remains robust to evolving patterns in the images, ensuring that its performance remains consistent even as the underlying data shifts.

### 4.3. Human Verification and Inter-Rater Reliability

Human experts may re-evaluate the sampled data to verify the LLM's judgments. Calculating inter-rater reliability metrics, such as F1 score as demonstrated during the pre-production analysis of this proposal, provides insights into the consistency between human and LLM evaluations, highlighting areas for improvement in the model or evaluation criteria. For example, discrepancies in the evaluation of ambiguous fields may indicate a need for updated prompts or additional training data to improve the LLM's contextual understanding.

### 4.4. Feedback Loop and Model Retraining

Feedback from human evaluations may be systematically fed back into the system, informing prompt adjustments and potential retraining of the LLM. For example, if a new document layout becomes prevalent, the feedback loop ensures that the system incorporates this layout into its training dataset and adapts accordingly. This iterative process ensures the model adapts to evolving data patterns and maintains high accuracy over time.

### 4.5. Statistical Tools for Monitoring

Advanced statistical tools, such as control charts and time-series analysis, may be employed to monitor trends and detect shifts in performance metrics. Control charts, for example, can identify instances where extraction accuracy falls outside predefined control limits, signaling the need for investigation. Time-series analysis provides insights into longer-term trends, helping distinguish between random fluctuations and systematic performance issues. These tools aid in identifying systematic issues versus random fluctuations, guiding targeted interventions.

### 5. Post-Production Scenario

The following scenario illustrates a theoretical application of the methodology described in the present disclosure. While theoretical, this scenario typifies how the disclosed system and methods would operate in a post-production environment to ensure continued robustness and effectiveness. The evaluation process builds upon the pre-production assessments by adding layers of continuous monitoring and periodic evaluation to maintain high performance standards in a live operational setting.

### 5.1. Evaluation Process

A similar sample size of 374 documents for a slightly smaller population of images is periodically evaluated by both human evaluators and the LLM-as-Judge. In this phase, 340 documents (approximately 91%) were correctly processed, indicating a slight improvement in accuracy. The 95% confidence interval for the true proportion of correctly processed documents is calculated as:

$$\hat{p} \pm M E = 0.91 \pm 0.0298 = ( 0.8802 , 0.9398 ) \qquad ( 14 )$$

This interval indicates that the system's accuracy, in a live production environment, is expected to remain within the range of 88.02% to 93.98%. This evaluation demonstrates that the system's performance is both reliable and consistent under operational conditions. The periodic reassessment ensures that any emerging data characteristics or operational challenges are identified and addressed promptly.

### 5.2. Agreement Between Evaluators

Consistent with pre-production findings, the agreement between human evaluators and the LLM-as-Judge remains high at 95% and an F1 score is derived. This consistency underscores the reliability of the LLM-as-Judge as a scalable evaluation tool in the post-production environment.

### 5.3. Overall System Effectiveness

Beyond the extraction of key/value pairs, the system's effectiveness includes the seamless integration of the extraction process into the broader business workflow, such as health benefit claims processing. Sampling results indicate that 91% of documents are processed correctly end-to-end, highlighting the reliability and operational efficiency of the system.

This disclosure presents a robust statistical framework for utilizing LLMs as evaluative tools in AI-driven key/value pair extraction tasks from complex images. By implementing the LLM-as-Judge approach alongside heuristic and human evaluators, we established a comprehensive methodology for both pre-production and post-production assessments. The high agreement rates between human evaluators and the LLM-as-Judge demonstrate the model's reliability and scalability for quality assessment. Continuous monitoring and iterative feedback loops further ensure sustained system performance and adaptability to evolving data patterns. The findings indicate that vision-enabled LLMs can effectively and consistently perform key/value pair extraction, offering significant benefits for applications in document processing and data automation. Future work may explore the scalability of this approach across different domains, the integration of additional evaluation metrics, and the application of alternative LLM architectures to enhance performance and versatility.

An example computer system in respect of which the methodology described above may be implemented is presented as a block diagram in FIG. 12. The example computer system is denoted generally by reference numeral 1200 and includes a display 1202, input devices in the form of keyboard 1204a and pointing device 1204b, computer 1206 and external devices 1208. While pointing device 1204b is depicted as a mouse, it will be appreciated that other types of pointing device, or a touch screen, may also be used.

The computer 1206 may contain one or more processors or microprocessors, such as a central processing unit (CPU) 1210. The CPU 1210 performs arithmetic calculations and control functions to execute software stored in a non-transitory internal memory 1212, preferably random access memory (RAM) and/or read only memory (ROM), and possibly storage 1214. The storage 1214 is non-transitory may include, for example, mass memory storage, hard disk drives, optical disk drives (including CD and DVD drives), magnetic disk drives, magnetic tape drives (including LTO, DLT, DAT and DCC), flash drives, program cartridges and cartridge interfaces such as those found in video game devices, removable memory chips such as EPROM or PROM, emerging storage media, such as holographic storage, or similar storage media as known in the art. This storage 1214 may be physically internal to the computer 1206, or external as shown in FIG. 12, or both. The storage 1214 may also comprise a database for storing images and data generated as a result of performing OCR on those images, as described above.

The one or more processors or microprocessors are examples of suitable processing units. Additionally or alternatively, a suitable processing unit may comprise any one or more of an artificial intelligence accelerator, programmable logic controller, a microcontroller (which comprises both a processing unit and a non-transitory computer readable medium), AI accelerator, or system-on-a-chip (SoC). As an alternative to an implementation that relies on processor-executed computer program code, a hardware-based implementation may be used. For example, other types of processing units such as an application-specific integrated circuit (ASIC), field programmable gate array (FPGA), or other suitable type of hardware implementation may be used as an alternative to or to supplement an implementation that relies primarily on a processor executing computer program code stored on a computer medium.

Any one or more of the methods described above may be implemented as computer program code and stored in the internal memory 1212 and/or storage 1214 for execution by the one or more processors or microprocessors to effect neural network pre-training, training, or use of a trained network for inference.

The computer system 1200 may also include other similar means for allowing computer programs or other instructions to be loaded. Such means can include, for example, a communications interface 1216 which allows software and data to be transferred between the computer system 1200 and external systems and networks. Examples of communications interface 1216 can include a modem, a network interface such as an Ethernet card, a wireless communication interface, or a serial or parallel communications port. Software and data transferred via communications interface 1216 are in the form of signals which can be electronic, acoustic, electromagnetic, optical or other signals capable of being received by communications interface 1216. Multiple interfaces, of course, can be provided on a single computer system 1200.

Input and output to and from the computer 1206 is administered by the input/output (I/O) interface 1218. This I/O interface 1218 administers control of the display 1202, keyboard 1204a, external devices 1208 and other such components of the computer system 1200. The computer 1206 also includes a graphical processing unit (GPU) 1220. The latter may also be used for computational purposes as an adjunct to, or instead of, the CPU 1210, for mathematical calculations.

The external devices 1208 include a microphone 1226, a speaker 1228 and a camera 1230. Although shown as external devices, they may alternatively be built in as part of the hardware of the computer system 1200. For example, the camera 1230 may be used to generate images of claims, following which OCR is performed.

The various components of the computer system 1200 are coupled to one another either directly or by coupling to suitable buses.

The term “computer system”, “data processing system” and related terms, as used herein, is not limited to any particular type of computer system and encompasses servers, desktop computers, laptop computers, networked mobile wireless telecommunication computing devices such as smartphones, tablet computers, as well as other types of computer systems.

The embodiments have been described above with reference to flow, sequence, and block diagrams of methods, apparatuses, systems, and computer program products. In this regard, the depicted flow, sequence, and block diagrams illustrate the architecture, functionality, and operation of implementations of various embodiments. For instance, each block of the flow and block diagrams and operation in the sequence diagrams may represent a module, segment, or portion of code, which comprises one or more executable instructions for implementing the specified action(s). In some alternative embodiments, the action(s) noted in that block or operation may occur out of the order noted in those figures. For example, two blocks or operations shown in succession may, in some embodiments, be executed substantially concurrently, or the blocks or operations may sometimes be executed in the reverse order, depending upon the functionality involved. Some specific examples of the foregoing have been noted above but those noted examples are not necessarily the only examples. Each block of the flow and block diagrams and operation of the sequence diagrams, and combinations of those blocks and operations, may be implemented by special purpose hardware-based systems that perform the specified functions or acts, or combinations of special purpose hardware and computer instructions.

The terminology used herein is for the purpose of describing particular embodiments only and is not intended to be limiting. Accordingly, as used herein, the singular forms “a”, “an”, and “the” are intended to include the plural forms as well, unless the context clearly indicates otherwise. It will be further understood that the terms “comprises” and “comprising”, when used in this specification, specify the presence of one or more stated features, integers, steps, operations, elements, and components, but do not preclude the presence or addition of one or more other features, integers, steps, operations, elements, components, and groups. Directional terms such as “top”, “bottom”, “upwards”, “downwards”, “vertically”, and “laterally” are used in the following description for the purpose of providing relative reference only, and are not intended to suggest any limitations on how any article is to be positioned during use, or to be mounted in an assembly or relative to an environment. Additionally, the term “connect” and variants of it such as “connected”, “connects”, and “connecting” as used in this description are intended to include indirect and direct connections unless otherwise indicated. For example, if a first device is connected to a second device, that coupling may be through a direct connection or through an indirect connection via other devices and connections. Similarly, if the first device is communicatively connected to the second device, communication may be through a direct connection or through an indirect connection via other devices and connections.

Use of language such as “at least one of X, Y, and Z,” “at least one of X, Y, or Z,” “at least one or more of X, Y, and Z,” “at least one or more of X, Y, and/or Z,” or “at least one of X, Y, and/or Z,” is intended to be inclusive of both a single item (e.g., just X, or just Y, or just Z) and multiple items (e.g., {X and Y}, {X and Z}, {Y and Z}, or {X, Y, and Z}). The phrase “at least one of” and similar phrases are not intended to convey a requirement that each possible item must be present, although each possible item may be present.

It is contemplated that any part of any aspect or embodiment discussed in this specification can be implemented or combined with any part of any other aspect or embodiment discussed in this specification, so long as such those parts are not mutually exclusive with each other.

The scope of the claims should not be limited by the embodiments set forth in the above examples, but should be given the broadest interpretation consistent with the description as a whole.

It should be recognized that features and aspects of the various examples provided above can be combined into further examples that also fall within the scope of the present disclosure. In addition, the figures are not to scale and may have size and shape exaggerated for illustrative purposes.

1. A method for extracting a key/value pair from an image file, the method comprising:
receiving the image file, wherein the image file comprises an input image representative of a document, and wherein the input image depicts one or more key/value pairs, wherein each of the key/value pairs comprises a key and an associated value as content within the document;
inputting the input image to one or more large language models (LLMs);
generating an image quality score indicating suitability of the input image for Optical Character Recognition (OCR) by inputting an image quality prompt comprising one or more image quality criteria to the one or more LLMs;
determining that the image quality score satisfies an image quality threshold;
applying OCR to the input image to obtain OCR output data comprising textual and structural features;
classifying the input image into a document type selected from a plurality of predetermined types by inputting a classification prompt including the textual and structural features to the one or more LLMs;
extracting the one or more key/value pairs from the OCR output data by inputting an extraction prompt including an extraction rule determined by the classified document type to the one or more LLMs; and
generating a confidence score for the one or more key/value pairs by inputting an evaluation prompt to the one or more LLMs, the evaluation prompt including one or more textual evaluation criteria including visual consistency between the one or more key/value pairs and part of the input image containing the one or more key/value pairs.

2. The method of claim 1 further comprising:
arranging the one or more key/value pairs and the confidence score in a structured document; and
storing the structured document.

3. The method of claim 1, wherein the one or more image quality criteria comprise at least one of text legibility, image sharpness, contrast, noise level, or text alignment.

4. The method of claim 1, wherein the image quality score is an average of the image quality scores corresponding to the one or more image quality criteria.

5. The method of claim 1, wherein the document is a medical-related document, and the plurality of predetermined types includes one or more of an invoice, a prescription, or a claim form.

6. The method of claim 1, wherein the textual and structural features of the OCR output data comprise at least one of a keyword or a layout element identified within the OCR output data.

7. The method of claim 6, wherein the layout element comprises one or more of a checkbox, a table, a header, a section break, or an angle of rotation of the input image.

8. The method of claim 1, wherein the confidence score is an average of confidence scores associated with all of the key/value pairs.

9. The method of claim 1, further comprising, removing the key/value pair having a blank value field.

10. The method of claim 1, further comprising preprocessing the input image before applying the OCR by performing at least one of cropping, rotating, resizing, or enhancing a contrast of the input image.

11. The method of claim 10, wherein the preprocessing is performed before generating the image quality score.

12. The method of claim 1, wherein the one or more textual evaluation criteria further comprise relevance between the one or more key/value pairs and the classified document type, wherein the method further comprises determining the relevance by assessing whether the extracted key/value pairs correspond to fields expected for the classified document type based on a predefined rule.

13. The method of claim 12, wherein the one or more textual evaluation criteria further comprise visual clarity, the visual clarity being derived from the image quality score for the part of the input image containing the one or more key/value pairs.

14. The method of claim 13, wherein the one or more textual evaluation criteria comprise weights applied to the visual consistency, the relevance, and the image clarity such that the confidence score is a weighted evaluation of the textual evaluation criteria.

15. The method of claim 14, wherein the weights applied to the visual consistency, the relevance, and the image clarity are 50%, 25%, and 25%, respectively.

16. The method of claim 1, wherein the image quality prompt comprises:
the one or more image quality criteria and corresponding definitions; and
a natural language request to estimate an image quality of the input image on a numerical scale as the image quality score.

17. The method of claim 1, wherein the classification prompt comprises:
the textual and structural features;
the plurality of predetermined types;
the one or more classification criteria and corresponding definitions; and
a natural language request to classify the input image into one of the plurality of predetermined types based on the textual and structural features.

18. The method of claim 1, wherein the extraction prompt further comprises:
guidance for handling different types of the textual and structural features; and
a natural language request to format the extracted the one or more key/value pairs into a structured output.

19. The method of claim 1, wherein the evaluation prompt comprises:
the one or more key/value pairs;
the one or more textual evaluation criteria and corresponding definitions; and
a natural language request to evaluate the one or more key/value pairs on a numerical scale as the confidence score.

20. The method of claim 1, wherein the generation of the image quality score, the classification of the input image, and the extraction of the one or more key/value pairs are performed by a first LLM, and wherein the generation of the confidence score is performed by a second LLM, distinct from the first LLM, the second LLM acting as a judge.

21. A non-transitory computer-readable medium storing computer-executable instructions that, when executed by one or more processing units, cause the one or more processing units to perform a method for extracting a key/value pair from an image file, the method comprising:
receiving the image file, wherein the image file comprises an input image representative of a document, and wherein the input image depicts one or more key/value pairs, wherein each of the key/value pairs comprises a key and an associated value as content within the document;
inputting the input image to one or more large language models (LLMs);
generating an image quality score indicating suitability of the input image for Optical Character Recognition (OCR) by inputting an image quality prompt comprising one or more image quality criteria to the one or more LLMs;
determining that the image quality score satisfies an image quality threshold;
applying OCR to the input image to obtain OCR output data comprising textual and structural features;
classifying the input image into a document type selected from a plurality of predetermined types by inputting a classification prompt including the textual and structural features to the one or more LLMs;
extracting the one or more key/value pairs from the OCR output data by inputting an extraction prompt including an extraction rule determined by the classified document type to the one or more LLMs; and
generating a confidence score for the one or more key/value pairs by inputting an evaluation prompt to the one or more LLMs, the evaluation prompt including one or more textual evaluation criteria including visual consistency between the one or more key/value pairs and part of the input image containing the one or more key/value pairs.

22. A system for processing an input image of a document, the system comprising:
a processing unit;
a memory communicatively coupled to the processing unit, the memory storing computer-executable instructions that, when executed by the processing unit, cause the processing unit to perform a method for extracting a key/value pair from an image file, the method comprising:
receiving the image file, wherein the image file comprises an input image representative of a document, and wherein the input image depicts one or more key/value pairs, wherein each of the key/value pairs comprises a key and an associated value as content within the document;
inputting the input image to one or more large language models (LLMs);
generating an image quality score indicating suitability of the input image for Optical Character Recognition (OCR) by inputting an image quality prompt comprising one or more image quality criteria to the one or more LLMs;
determining that the image quality score satisfies an image quality threshold;
applying OCR to the input image to obtain OCR output data comprising textual and structural features;
classifying the input image into a document type selected from a plurality of predetermined types by inputting a classification prompt including the textual and structural features to the one or more LLMs;
extracting the one or more key/value pairs from the OCR output data by inputting an extraction prompt including an extraction rule determined by the classified document type to the one or more LLMs; and
generating a confidence score for the one or more key/value pairs by inputting an evaluation prompt to the one or more LLMs, the evaluation prompt including one or more textual evaluation criteria including visual consistency between the one or more key/value pairs and part of the input image containing the one or more key/value pairs.
